(19) United States
(12) Patent Application Publication
(10) Pub. No.: US 2022/0172641 A1
(43) Pub. Date: Jun. 2, 2022

(54) SYSTEMS AND METHODS FOR IMPROVING PERFORMANCE VIA MENTAL TRAINING

(71) Applicant: Apeak Corporation, Kenmore, WA (US)

(72) Inventor: Hung Soon Park, Kenmore, WA (US)

(21) Appl. No.: 17/338,180

(22) Filed: Jun. 3, 2021

Related U.S. Application Data
(60) Provisional application No. 62/704,938, filed on Jun. 3, 2020.

(51) Int. Cl. G09B 19/00; G09B 5/06; G09B 5/04; G16H 20/30; G16H 20/70

(52) U.S. Cl. CPC G09B 19/00; G09B 5/065; G16H 20/70; G16H 20/30; G09B 5/04

(57) ABSTRACT

Systems, devices, and methods for improving mental skills and/or automatically processing user input for purposes of improving mental skills are disclosed herein. In some embodiments, a method of mental training can include (i) providing to a user a pre-module that includes a plurality of pre-tagged first objects; (ii) receiving, in response to the pre-module, a user input including a plurality of second objects; (iii) processing the second objects of the user input by comparing the second objects to the first objects to determine a correlation; and (iv) after processing the second objects, automatically determining a post-module to be provided to the user based on the correlation.

### CROSS-REFERENCE TO RELATED APPLICATION(S)

The present application claims the benefit of and priority to U.S. Provisional Patent Application No. 62/704,938, filed Jun. 3, 2020, the disclosure of which is incorporated herein by reference in its entirety.

### TECHNICAL FIELD

The present technology relates to processing user inputs to provide automated mental training and associated systems, devices and methods.

### BACKGROUND

Athletic performance, or performance generally, is closely tied to mental fitness and skill. Mental aptitude and training for athletes, professionals, and the like is a key component of being able to compete and perform at the highest levels. For example, developing an individual's ability to maintain consistency during performance, have the right attitude regardless of external factors, and understand an individual's ideal performance state, amongst other abilities, are instrumental to achieving optimal performance for that individual. However, despite the importance of mental training, few individuals receive the necessary training to realize their peak performance levels. This is largely due to the high cost of mental training and the limited availability (e.g., time availability and geographic availability) of mental training coaches. The high cost and limited availability of mental training can be at least partially due to the requirement for human-to-human interaction and/or the lack of technological tools able to provide adequate mental training exercises. As such, there exists a general need to provide improved systems and methods for enhancing performance via mental training.

### BRIEF DESCRIPTION OF THE DRAWINGS

Many aspects of the present disclosure can be better understood with reference to the following drawings.

FIG. 1 is a schematic block diagram of a computing device in which a system for determining and/or improving performance via mental training may operate, in accordance with embodiments of the present technology.

FIG. 2 is a schematic block diagram illustrating a suitable environment in which the disclosed system may be implemented, in accordance with embodiments of the present technology.

FIG. 3 is a flow diagram illustrating a process for improving performance via mental training, in accordance with embodiments of the present technology.

FIG. 4 is a flow diagram illustrating a process for automatically processing a user input for purposes of improving mental skills, in accordance with embodiments of the present technology

FIGS. 5A-5C are example screenshots of the software or application in use on a mobile device, in accordance with embodiments of the present technology.

### DETAILED DESCRIPTION

Mental training for athletes, professionals, and the like is a key component of being able to compete and perform at the highest levels. For example, developing an individual's ability to maintain consistency during performance, have the right attitude regardless of external factors, and understand an individual's ideal performance state, amongst other tasks, are instrumental to achieving optimal performance for that individual. However, despite the importance of mental training, few individuals receive the necessary training to realize their peak performance levels. This is largely due to the cost of mental training and the limited availability (e.g., time availability and geographic availability) of mental training coaches. Additionally, attempts to automate mental training, e.g., via computerized systems, have been generally unsuccessful, in part because such systems have been unable to (i) tailor the training to a particular individual, (ii) enable individuals to focus on and improve their mental weaknesses, and/or (iii) continuously update based on the particular individual's mental growth and development. As such, there exists a general need to provide improved systems and methods for enhancing performance via mental training.

The present technology relates to systems and methods for improving performance via mental training, and mitigates some of the previously described issues. Some embodiments of the present technology, for example, are directed to improving a user's mental strength by exposing the user to a set of mental test modules, determining exercise modules based on the user's response to the mental test modules, and monitoring the user's performance of the exercise modules. The test modules may be directed to certain aspects of mental aptitude, such as confidence, attitude, managing performance expectations, breathing skill, ability to face adversity, and the like. Moreover, the test modules may be specific to a particular activity such as sports (e.g., tennis, basketball, golf, etc.). Based on the monitored performance of the user and/or inputs from the user, additional test modules can be automatically provided to the user, e.g., to continuously improve the user's mental skill.

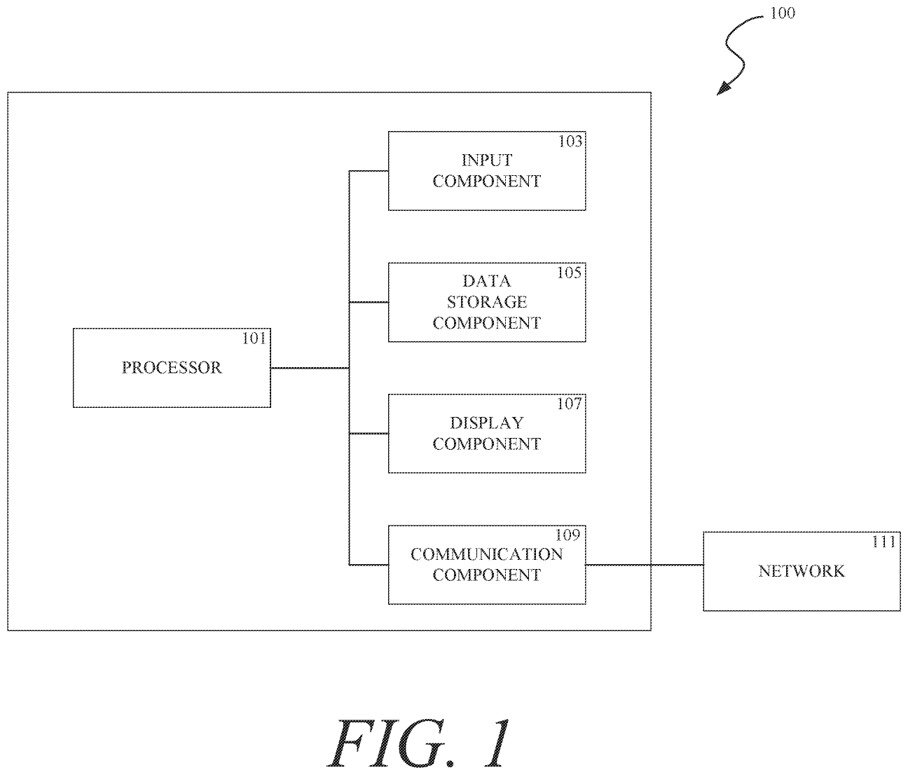

Some embodiments of the present technology are directed to methods, systems, and/or devices for automatically processing a user input for purposes of improving mental skills. The method can comprise providing to a user a pre-module that includes a plurality of pre-tagged first objects, receiving a user input, in response to the pre-module provided to the user, that includes a plurality of second objects, processing the second objects of the user input by comparing the second objects to the first objects to determine a correlation, and, after processing the second objects, automatically determining a post-module to be provided to the user based on the correlation. Specific details of several embodiments of the technology are described below with reference to FIGS. 1-5C.

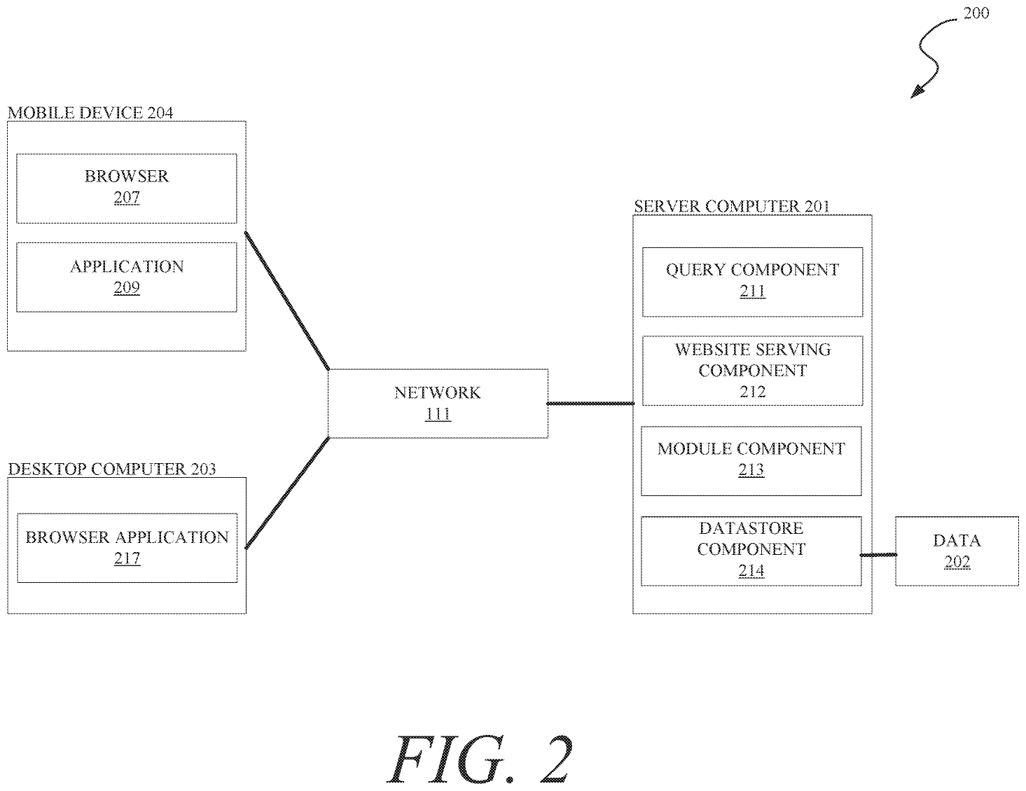

FIGS. 1 and 2 and the following discussion provide a brief, general description of suitable computing environments in which aspects of the present technology can be implemented. Although not required, aspects and embodiments of the present technology will be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, mini-computers, mainframe computers, and the like. The present technology can be embodied in a special purpose computer or data processor that is specifically programmed, configured, or constructed to perform one or more of the computer-executable instructions explained in detail below. In some embodiments, the disclosed functionality may be implemented by instructions encoded in a non-transitory computer-readable storage medium.

The present technology can also be practiced in distributed computing environments, where tasks or modules are performed by remote processing devices, which are linked through a communications network, such as a Local Area Network (“LAN”), Wide Area Network (“WAN”) or the Internet. In a distributed computing environment, program modules or sub-routines may be located in both local and remote memory storage devices. Aspects of the present technology described below can be stored or distributed on computer-readable media, including magnetic and optically readable and removable computer discs, stored as firmware in chips (e.g., Electrically-Erasable Programmable Read-Only Memory chips, EEPROM chips), as well as distributed electronically over the Internet or over other networks (including wireless networks). Those skilled in the relevant art will recognize that portions of the present technology can reside on a server computer, while corresponding portions reside on a mobile device. Data structures and transmission of data particular to aspects of the present technology are also encompassed within the scope of the present technology.

FIG. 1 is a schematic block diagram illustrating components of a computing device 100, such as a desktop computer, smartphone, tablet computer, laptop, wearable computer, etc., in which the interface for improving performance via mental training may be generated. As shown in FIG. 1, the computing device 100 includes a processor 101, an input component 103, a data storage component 105, a display component 107, and a communication component 109. The processor 101 is configured to couple with and control other components in the computing device 100. The computing device 100 can communicate with other systems (e.g., Web servers or other devices) through the communication component 109 via a network 111. In some embodiments, the computing device 100 can communicate with an output device (e.g., printers, speakers, tactile output devices, etc.) through the communication component 109. The network 111 can be any private or public network, such as the Internet, a corporate intranet, a wireless communication network, or a wired communication network.

The input component 103 is configured to receive an input (e.g., an instruction or a command) from a device user. The input component 103 can include a keyboard, a mouse, a touch pad, a touchscreen, a microphone, a pen, a scanner, a camera, and/or the like. The data storage component 105 can include any type of computer-readable media that can store data accessible to the processor 101. In some embodiments, the data storage component 105 can include random-access memories (RAMs), read-only memories (ROMs), flash memory cards, magnetic hard drives, optical disc drives, digital video discs (DVDs), cartridges, smart cards, etc.

The display component 107 is configured to display information to the mobile device user. In some embodiments, the display component 107 can include flat panel displays such as liquid crystal displays (LCDs), light emitting diode (LED) displays, plasma display panels (PDPs), electro-luminescence displays (ELDs), vacuum fluorescence displays (VPDs), field emission displays (FEDs), organic light emitting diode (OLED) displays, surface conduction electron emitter displays (SEDs), or carbon nano-tube (CNT) displays.

FIG. 2 is a schematic block diagram of an environment 200 in which the system for generating an interface for improving performance via mental training may operate. The environment 200 can include one or more server computers 201 that access data stores 202 containing information associated with mental training. For example, as explained elsewhere herein, the data stores 202 may include mental aptitude test scores for students, including the demographic information (e.g., age, gender, etc.) of the students. The server computers 201 communicate with computing devices 100 via a network 205. The computing devices 100 may send search queries to the server computers 201 pertaining to the unique items. The search queries are processed by the server computers 201 against the data in the data stores 202. The server computers 201 may retrieve, analyze, and/or format (e.g., in datasets) unique item information that is responsive to the received search queries. The server computer 201 transmits data responsive to the search queries to a requesting computing device 100 through the network 205. The network 205 can include the Internet, an intranet, a wireless communication, or a wired communication.

The server computer 201 includes a query processing component 211, a website serving/management component 212, a module component 213, and a datastore or database management component 214. The query component 211 is configured to perform query processing and analysis. The website management component 212 is configured to handle creation, display and/or routing of suitable information in the form of web pages. The module component 213 is configured to serve sets of modules as described herein in a manner that enables determination of the user's mental aptitude for different categories. The module component 213 may be separate from, or incorporated within, the website management component 212. The database management component 214 is configured to manage access to and maintenance of data stores 202. The server computer 201 can employ security measures (e.g., firewall systems, secure socket layers (SSL), password protection schemes, encryption, and/or the like) to inhibit malicious attacks and to preserve integrity of the information stored in the data stores 202.

The computing device 100 may include one or more programs that submit queries to the server computers and receive responsive results. For example, a browser application 207 on a mobile device 204 is configured to access and exchange data with the server computer 201 through the network 205. Results of data queries may be displayed in the mobile device 204 or a browser (e.g., Firefox, Chrome, Internet Explorer, Safari, etc.) of the mobile device 204 for viewing by the device user. Similarly, a browser application 217 on a desktop computer 203 is configured to access and exchange data with the server computer 201 through the network 205, and the results of the data queries may be displayed in the browser for review by the device user. As another example, a dedicated application 209 on the mobile device 204 is configured to display or present received information to a mobile device user via the application. Data may be received from the server computer 201 via an application programming interface (API), and the received data formatted for display by the application on the computing device 100. The server computer 201 and the computing device 100 can include other programs or modules such as an operating system, one or more productivity application programs (e.g., word processing or spread sheet applications), and the like

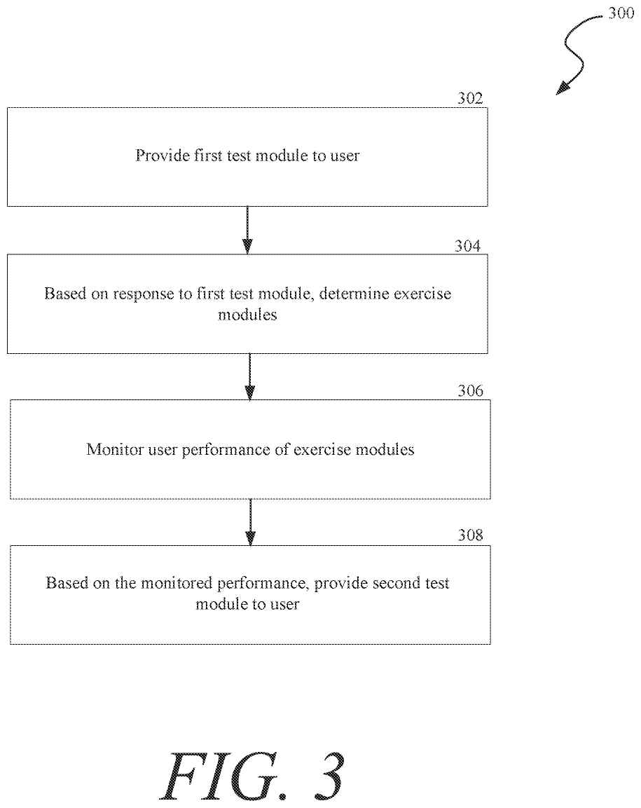

FIG. 3 is a flow diagram illustrating a process 300 for improving performance via mental training, in accordance with embodiments of the present technology. The process 300 can include providing one or more first test modules (e.g., pre-modules) to a user (process portion 302). The first test modules can be based on aspects of mental aptitude or ability for performing at an optimal level. For example, the first test modules may relate to categories including: (i) confidence, (ii) attitude, (iii) managing performance expectations, (iv) understanding an ideal performance state, (v) maintaining composure during performance, (vi) ability to create positive energy, (vii) ability to visualize, (viii) tactical awareness, (ix) performance during critical times, (x) breathing skill, and (xi) ability to face adversity for the user. Each of the first test modules can include or be associated with questions meant to test or gauge the user's aptitude for the previously described mental aptitude aspects. For example, the test module for confidence may ask the user to answer (i) whether the user has doubts about his performance ability, and/or (ii) whether confidence in performance varies based on an opponent or the likelihood of winning. The user can respond to the questions for each of the test modules by selecting one of multiple available answers and/or providing a typed, audio, or video input, e.g., via a mobile device. The responses from the user for each of the first test modules may be transcribed and/or translated into a numerical score or value that can be used to determine whether the user is below, at, or above a predetermined threshold for that particular test module. Translation of a response to a numerical score may be done via an algorithm based at least in part on the user's history and current status for a particular test module. For example, depending on where along a training program the user currently is, each answer may correspond to a different numerical score. The predetermined threshold for each test module may be dynamic, and/or determined based on the characteristics (e.g., age, gender, etc.) of the user and/or the category of performance (e.g., athletics). For example, the predetermined threshold for the test module for confidence may be different from females aged 10-12 than it is for males aged 12-14.

In some embodiments, the method 300 can include providing a baseline test to the user. The baseline test may include a number of questions to help the system gauge the user's relative strengths and weaknesses for each of the categories previously described. In such embodiments, the first test module (and subsequent test modules) provided to the user may be based on the baseline test provided to the user. Additionally or alternatively, the baseline test may be used throughout the method 300 to determine the predetermined threshold for each module.

The method 300 can further comprise determining exercise modules based on the responses to the first test modules (process portion 304). That is, the determined exercise module may vary depending on the user's response and whether the numerical score associated with the response is above the predetermined threshold. The exercise modules can include instructions or activities related to the categories previously described that the user is to perform on or separate from the user's mobile device. For example, the exercise modules can include performing visualizations and/or physical exercises, meditating, watching audio and/or video content, and/or other activities meant to improve the user's abilities for the particular category of the exercise module. Each of the exercise modules may be directed to a particular event, such as tennis, golf, basketball, baseball, football, or the like. In such embodiments, the visualizations, physical exercises, meditations, and the like are specific to one event (e.g., tennis) and are different than the respective visualizations, physical exercises, and meditations for other events (e.g., golf, basketball, baseball, football, etc.) As a more specific example, the exercise module directed to breathing skill may instruct the user to perform breathing exercises while listening to audio content provided by the mobile device/program.

The method 300 can further comprise monitoring the user's performance of the exercise modules (process portion 306). Monitoring the user's performance can include recording, e.g., via a camera or other component(s) of the user's mobile device, the user performing the exercise modules, and/or storing the user's responses to the exercise modules. In some embodiments, the recorded and/or stored information can be transmitted to a mentor, coach, or other individual assigned to the user. In such embodiments, the mentor (or other individual) can review the performance and determine subsequent exercise modules for the user to perform. Additionally or alternatively, the user's performance may be reviewed and/or analyzed by software utilizing artificial intelligence (AI), e.g., to make an initial determination whether the user's performance was adequately completed. Examples of suitable AI-based approaches to analyzing the user's performance include machine learning, deep learning (e.g., neural network models), or other suitable techniques. Based on the review and/or analysis, a subsequent exercise module may be suggested to a mentor (or other individual) and/or provided directly to the user to complete.

The method 300 can further comprise providing a second test module (e.g., post-test modules) to the user based at least in part on the monitored performance (process portion 308). The second test module can correspond to any of the test modules previously described and may be different than the first module previously provided to the user via process portion 302. For example, the second test module can relate to categories including: (i) confidence, (ii) attitude, (iii) managing performance expectations, (iv) understanding and ideal performance state, (v) maintaining composure during performance, (vi) ability to create positive energy, (vii) ability to visualize, (viii) tactical awareness, (ix) performance during critical times, (x) breathing skill, and (xi) ability to face adversity. Subsequent to providing the second test module to the user, process portions 304, 306, and 308 may be iteratively performed, e.g., until the user has completed all of the test modules and/or until the user has achieved a score above a predetermined threshold.

As previously mentioned, the benefits of mental training are often not realized by athletes or other individuals (collectively “students” or “users”) because of its excess cost and limited availability, which can be due to time availability and/or geographic availability (e.g., limited coaches available in a particular area). In part, this excess cost and limited availability are due to the amount of human interaction it takes for coaches to work with students to develop their mental fitness and strength. Additionally, the significant time coaches have previously had to invest in students was heightened because of the limited availability of technological tools that are able to both (i) facilitate feedback between the student and coach, and (ii) automatically assess a student's deficiencies.

Embodiments of the present technology attempt to mitigate these issues by developing systems, devices, methods, and/or software to automatically assess student or user input and respond with tailored instructions for the purposes of improving their mental skills. As an example, embodiments of the present technology can provide training modules (e.g., pre-modules) directed to different mental aptitude categories, as previously described, and which have intended goals for the user's mental development. The modules can seek to obtain specific user feedback regarding a particular category. Before requesting input from a particular user, certain objects (e.g., keywords) that may be expected to appear in a user's input can be tagged by the system. If there is enough of a correlation between the tagged words and those words inputted by the user, the system can recommend a subsequent training module or exercise (e.g., a post-module) meant to further improve the user's mental skill.

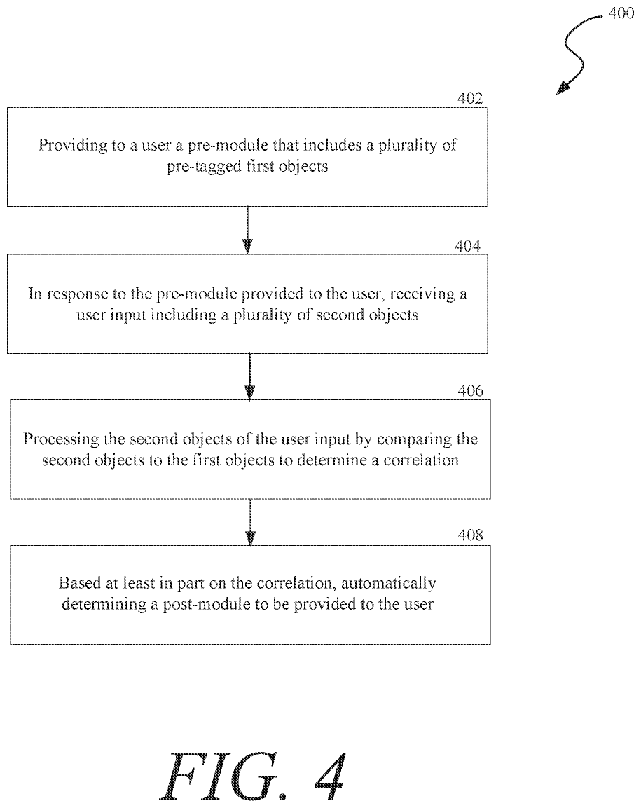

Additional details regarding such embodiments are provided in FIG. 4, which is a block flow diagram of a method 400 for automatically processing a user input for purposes of improving mental skills, e.g., with respect to sports. The method 400 can include providing to a user a pre-module that includes a plurality of pre-tagged first objects (process portion 402). The pre-modules can be, for example, a pre-training module that includes at least one of visualizations, audio instructions, video instructions, or other exercise recommendation for the user to perform. The pre-training module can be associated with a particular mental aptitude category (e.g., confidence, attitude, managing performance expectations, understanding an ideal performance state, maintaining composure during performance, ability to create positive energy, ability to visualize, tactical awareness, performance during critical times, breathing skill, ability to face adversity for the user, etc.), as described elsewhere herein.

In some embodiments and as explained in more detail below, the pre-module may have a plurality of pre-tagged first objects associated therewith. The pre-tagged first objects can be grouped into one or more subsets each associated with one or more post-modules. That is, a primary subset of the pre-tagged first objects can be associated with a first post-module, in that if a user's input includes objects that sufficiently correlate with the first objects, then a first post-module will be provided. Additionally, more subsets (e.g., a secondary subset) of the pre-tagged first objects can be associated with more post-modules (e.g., a second post-module) different than the first post-module. As an example of such modules related to tennis, a primary subset can include objects related to second serves, such as “second serves,” “double faults,” “critical,” “point missed,” “struggle,” and/or “missed,” and a secondary subset can include objects related to “referee,” “official,” “bad,” “line call,” “cheated,” and/or “hook.” As explained in more detail below, the primary subset can lead to a first post-module being automatically provided (e.g., without human involvement) to the user and configured to improve the user's second serve ability. The secondary subset can lead to a second post-module being automatically provided to the user and configured to improve the user's ability to deal with umpires/officials/referees and/or perceived bad calls. In doing so, the system can automatically address deficiencies of users to improve their ability with regard to a particular subject.

Once the pre-module is provided to the user, and/or once the user completes the trainings or exercises prescribed by the pre-module, the system may request feedback from the user for that pre-module. For example, if the pre-module is associated with the mental aptitude category of confidence, then the requested feedback may be related to confidence and ask how confident the user felt in their exercises or matches today. Alternatively, the requested feedback may not be associated with the pre-module. For example, the requested feedback may be more generic and ask what the user can improve based on his or her performance today. In response, the user provides a user input comprising a plurality of objects. As such, the method 400 can further comprise receiving, in response to the pre-module provided to the user, a user input including a plurality of second objects (process portion 404). In some embodiments, the second objects comprise keywords entered electronically (e.g., via a mobile application), or an audio or video recording. Embodiments including audio, including video, can be transcribed by the system and then processed by the system as if the transcription was provided as keywords.

The method 400 can further comprise processing or analyzing the second objects of the user input by comparing the second objects to the first objects, e.g., to determine a correlation (process portion 406). Processing the second objects can include grouping certain objects, such as those immediately subsequent to one another together, if the grouped objects match any one of the first objects. The objects to be grouped by the system can be pre-tagged as grouped objects. For example, the terms “second” and “serve” inputted by a user can be grouped together to form a single object “second serve.” Additionally or alternatively, processing the second objects can include comparing individual ones of the second objects entered by the user to the pre-tagged objects for a particular post-module. In such embodiments, comparing the second objects and the pre-tagged objects can include determining a number and/or percentage of pre-tagged objects for a particular post-module that are included in the first objects entered by the user. For example, if five out of a total of ten pre-tagged objects for a particular post-module are entered by the user as first objects, then a 50% correlation can be assigned to the first objects for the particular post-module associated with the pre-tagged objects. As another example, if at least a predetermined number (e.g., three, four, five, six, etc.) pre-tagged objects for a particular post-module are entered by the user as first objects, then another correlation can be assigned to the first objects for the particular post-module associated with the pre-tagged objects. In some embodiments, certain ones of the first objects can be weighted higher than other ones of the first objects. For example, if the term “second serves” are included in the user input as first objects, the system may weigh that term higher than other terms included in the user input, such as “critical.” This is because the term “second serve” is likely only associated with a user indicating that he or she needs to work on their second serves, as opposed to the term “critical” which could indicate multiple deficiencies. In some embodiments, the first objects entered by the user can match and/or correlate to pre-tagged objects of more than one different post-modules. For examples, the first objects may have a 50% correlation with a first post-module, a 40% correlation with a second post-module, and a 60% correlation with a third post-module.

The method 400 can further comprise, based on the correlation, automatically determining a post-module to be provided to the user (process portion 408). Post-modules can be an exercise, instructions, and/or an audio or video visualization that is related to a particular category or deficiency identified by the user. The post-module can be meant to help improve the user's ability related to the category or deficiency. Automatically determining the post-module can be based at least in part on the correlation percentage previously described being at or above a predetermined threshold. The predetermined threshold can be 30%, 40%, 50%, 60%, 70%, any incremental value therebetween, or within a range of 30-70%. For example, if a correlation percentage and predetermined threshold are each 50%, then the corresponding post-module would be provided to the user. As previously described, multiple post-modules can be provided to the user based on a single user input of first objects. In some embodiments, the history of the user can be considered when determining whether to provide a post-module to that user. For example, if user had been previously provided a post-module associated with “second serves,” the predetermined threshold for providing the same post-module, or another similar post-module associated with “second serves,” may be lower than the predetermined threshold initially required when the post-module was previously provided. This may be done because the system already knows that “second serves” are an identified deficiency of the user.

In some embodiments, when the correlation percentage is below the predetermined threshold for any corresponding post-module, the system may generate an indication (e.g., a message, email, etc.) to a system administrator (e.g., a coach) to indicate that no post-modules could be determined based on the user input. The indication can include the user's input. In such embodiments, the system administrator can review the user's input, e.g., to recommend an appropriate post-module. Additionally or alternatively, when the correlation percentage is below the predetermined threshold for any corresponding post-module, the system may request additional feedback from the user. The additional feedback, in combination with the initial user input, can then be used to provide a post-module according to the method 400.

Embodiments of the present technology have multiple advantages for improving mental skills of users. As previously mentioned, mental training provided by experts (e.g., coaches, sports psychologists, and the like) can be expensive and not easily accessible to individuals not in physical close proximity to the experts. As such, many individuals are unable to receive the mental training needed to improve their performance in sports, management, or other industries. Embodiments of the present technology address this, e.g., by providing systems, devices, methods, and/or software able to provide training modules to users based on their own identified deficiencies, and to do so automatically in a manner that does not require the expert to personally address each deficiency. In doing so, the number of users able to receive effective mental training can be significantly increased. Stated differently, embodiments of the present technology enable the ability for mental training methods to be scaled to multiple users regardless of location.

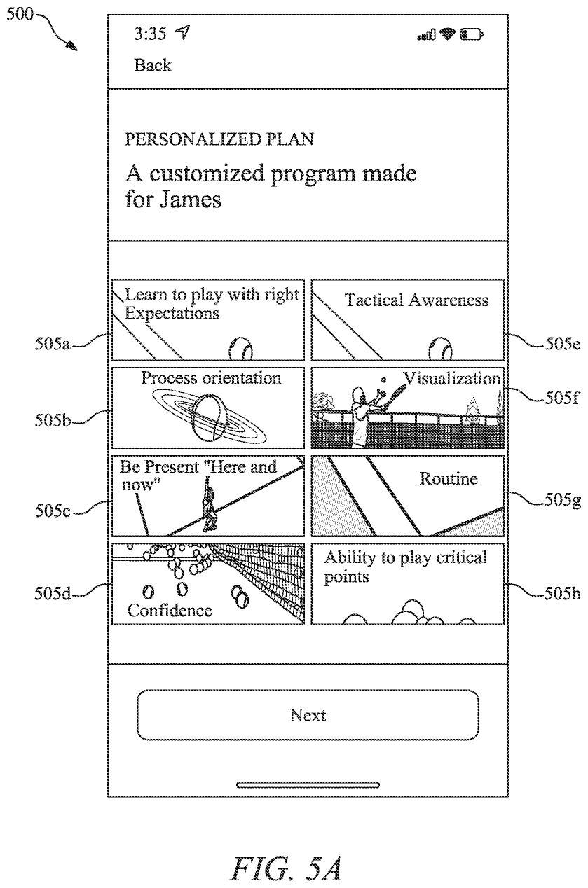

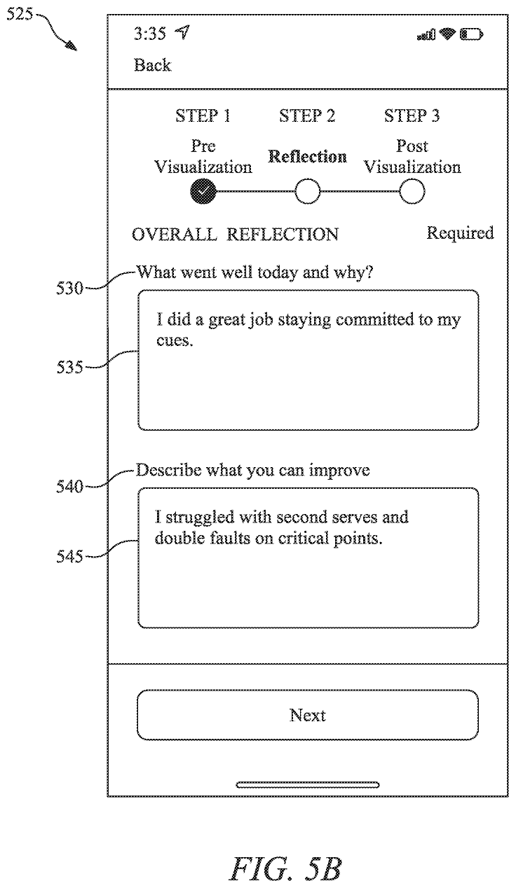

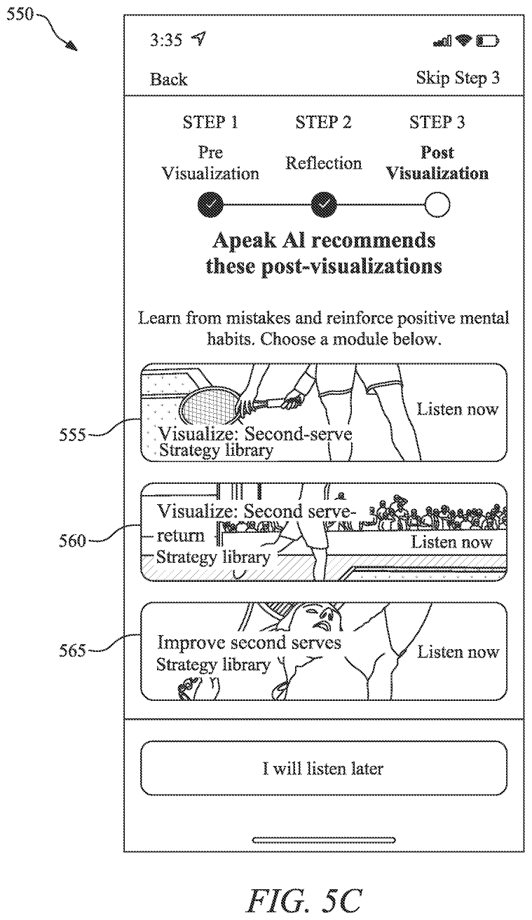

FIGS. 5A-5C include example screenshots of the present technology in use via an application on a mobile device. Referring first to FIG. 5A, the application can include an example screenshot 500 with a list of pre-training modules 505a-h (collectively referred to as “pre-modules 505”), which may be tailored to a particular user, e.g., based on the user's needs or user's baseline test score (as previously described). The list of pre-modules 505 can be prioritized based on the user's strengths and/or weaknesses, with the user's weaknesses having a higher priority. As shown in FIG. 5A, the pre-modules can include a “learning to play with the right expectations” module 505a, a “process orientation” module 505b, a “be present ‘here and now’” module 505c, a “confidence” module 505d, a “tactical awareness” module 505e, a “visualization” module 505f, a “routine” module 505g, and “an ability to play critical points” 505h, amongst other modules. As shown in FIG. 5A, the modules 505 are specific to tennis. In other embodiments, the modules 505 can specific to other sports (e.g., basketball, football, golf, baseball, etc.) or industries (e.g., management, leadership, etc.).

FIG. 5B is an example screenshot 525 in which one or more user inputs is requested. The screenshot 525 can be displayed to the user after one of the pre-modules are selected by the user and/or the exercises or instructions associated with the pre-module are completed. As shown in FIG. 5B, the screenshot 525 can include one or more questions 530, 540 and one or more user input boxes 535, 545. The questions 530, 540 can be associated with one of the pre-modules 505 shown and described in FIG. 5A, in that they are related to the category of one of the pre-modules 505. The user input entered in the boxes 535, 545 can be the second objects described elsewhere herein (e.g., with reference to FIG. 4), and can be analyzed and/or used to automatically determine a post-module to be provided to the user (e.g., process portions 406, 408). As described elsewhere herein, the objects of the user input 545 can be analyzed by comparing the objects entered by the user to pre-tagged objects (e.g., first objects; FIG. 4). As shown in the user input box 545 of FIG. 5B, the second objects, which include the terms “second serves” and “double faults” can be analyzed to produce a post-module associated with improving second serves.

FIG. 5C is an example screenshot 550 of a third module in which one or more post-modules 555, 560, 565 is provided to the user based on the objects provided on screenshot 525. As described elsewhere herein (e.g., with reference to FIG. 4), the post-modules 555, 560, 565 can be chosen and/or provided automatically by the system, without human intervention, based at least in part on the second objects entered on screenshot 525. Each of the post-modules 555, 560, 565 can include one or more exercises, audio instructions, and/or videos, and can be directed to improving a user deficiency, as determined by analyzing the second objects entered by the user in view of the pre-tagged objects associated with the pre-test module.

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.

Clause 1. A method for automatically processing a user input for purposes of improving mental skills, the method comprising:
providing a pre-module to a user, the pre-module including a plurality of pre-tagged first objects;
receiving a user input in response to the pre-module provided to the user, the user input including a plurality of second objects;
processing the second objects of the user input by comparing the second objects to the first objects to determine a correlation; and
after processing the second objects, automatically determining a post-module to be provided to the user based on the correlation.

Clause 2. The method of any one of the clauses herein, wherein the correlation corresponds to a percentage of the second objects that match the first objects.

Clause 3. The method of any one of the clauses herein, wherein automatically determining the post-module is based on the match percentage being above a predetermined threshold.

Clause 4. The method of any one of the clauses herein, wherein:
the pre-tagged first objects comprise (i) a subset of primary objects configured to produce a first post-module and (ii) a subset of secondary objects configured to produce a second post-module different than the first post-module,
processing the second objects comprises comparing the second objects to each of the primary objects and the secondary objects, and
automatically determining the post-module comprises automatically determining which of the first post-module or second post-module to provide to the user based on which of the primary objects or the secondary objects has a higher correlation with the second objects.

Clause 5. The method of any one of the clauses herein, wherein automatically determining the post-module is based at least in part on the order of individual second objects relative to one another in the user input.

Clause 6. The method of any one of the clauses herein, wherein the first objects and the second objects comprise keywords, and wherein the second objects are provided in response to a question provided via the pre-module.

Clause 7. The method of any one of the clauses herein, wherein the pre-module is one of a plurality of pre-modules including a first pre-module directed to a first mental aptitude category, and a second pre-module directed to a second mental aptitude category, the second mental aptitude category different than the first mental aptitude category.

Clause 8. The method of any one of the clauses herein, further comprising, after processing the second objects, if the comparison of the first objects and the second objects is below a predetermined threshold, then sending an indication of such to a system administrator.

Clause 9. The method of any one of the clauses herein, further comprising, after processing the second objects:
if the comparison of the first objects to the second objects is at or above a predetermined threshold, then sending the determined post-module to the user; and
if the comparison of the first objects and the second objects is below a predetermined threshold, then sending an indication of such to a system administrator.

Clause 10. The method of any one of the clauses herein, wherein the pre-module and the post-module are each directed to improving mental skills for a sport.

Clause 11. A method for automatically processing user feedback for purposes of developing mental skill, the method comprising:
providing a pre-module to a user;
receiving an indication that the user has completed the pre-module;
after receiving the indication, requesting a user input based at least in part on the provided pre-module;
receiving a user input in response to the pre-module provided to the user, the user input including a plurality of keywords;
processing the keywords of the user input by comparing the keywords to a plurality of pre-tagged keywords to determine a correlation; and
based on the determined correlation, automatically determining a post-module to be provided to the user.

Clause 12. The method of any one of the clauses herein, wherein the post-module is a recommended training instruction comprising at least one of video or audio.

Clause 13. The method of any one of the clauses herein, wherein the steps of providing the pre-module and receiving the user input occur via a mobile application.

Clause 14. The method of any one of the clauses herein, wherein processing the keywords of the user input comprises determining a percentage of the second objects that match the pre-tagged keywords, and wherein automatically determining the post-module is based on the determined percentage being above a predetermined threshold.

Clause 15. The method of any one of the clauses herein, further comprising, after processing the second objects:
if the comparison of the first objects to the second objects is at or above a predetermined threshold, then sending the determined post-module to the user; and
if the comparison of the first objects and the second objects is below a predetermined threshold, then sending an indication of such to a system administrator.

Clause 16. A device, comprising:
at least one processor; and
tangible, non-transitory computer-readable media having instructions stored therein, wherein the instructions, when executed by the at least one processor, cause the device to perform operations comprising:
   providing a pre-module to a user, the pre-module including a plurality of pre-tagged first objects;
   receiving a user input in response to the pre-module provided to the user, the user input including a plurality of second objects;
   processing the second objects of the user input by comparing the second objects to the first objects to determine a correlation; and
   after processing the second objects, automatically determining a post-module to be provided to the user based on the correlation.

Clause 17. The device of any one of the clauses herein, wherein the correlation corresponds to a percentage of the second objects that match the first objects, and wherein automatically determining the post-module is based on the match percentage being above a predetermined threshold.

Clause 18. The device of any one of the clauses herein, wherein:
the pre-tagged first objects comprise (i) a subset of primary objects configured to produce a first post-module and (ii) a subset of secondary objects configured to produce a second post-module different than the first post-module,
processing the second objects comprises comparing the second objects to each of the primary objects and the secondary objects, and
automatically determining the post-module comprises automatically determining which of the first post-module or second post-module to provide to the user based on which of the primary objects or the secondary objects has a higher correlation with the second objects.

Clause 19. The device of any one of the clauses herein, wherein automatically determining the post-module is based at least in part on the order of individual second objects relative to one another in the user input.

Clause 20. The device of any one of the clauses herein, wherein the post-module is a recommended training instruction comprising at least one of video or audio.

Clause 21. A method for mental training, the method comprising:
providing a first test module associated with a mental aptitude category;
based on a response to the first test module, determining and providing an exercise module;
monitoring a user's performance of the provided exercise module; and
based on the monitored performance, providing a second test module different than the first test module and associated with the mental aptitude category.

Clause 22. The method of any one of the clauses herein, wherein the mental aptitude category includes at least one of confidence, attitude, managing performance expectations, understanding an ideal performance state, maintaining composure during performance, ability to create positive energy, ability to visualize, tactical awareness, performance during critical times, breathing skill, or ability to face adversity.

Clause 23. The method of any one of the clauses herein, wherein providing the first test module comprises providing a set of questions related to mental aptitude category.

Clause 24. The method of any one of the clauses herein, wherein the exercise module is specific to a sport.

Clause 25. The method of any one of the clauses herein, wherein the exercise module is specific to tennis.

Clause 26. The method of any one of the clauses herein, further comprising receiving and translating the response for the first test module into a numerical value based on one or characteristics of the user, the characteristics including age and gender.

Clause 27. The method of any one of the clauses herein, wherein the provided exercise module is based on whether the numerical value is above a predetermined threshold.

Clause 28. The method of any one of the clauses herein, further comprising providing a baseline test to the user prior to providing the first test module, wherein the provided first test module is based on a response to the baseline test.

Clause 29. The method of any one of the clauses herein, wherein the exercise module comprises instructions to perform a visualization, a physical exercise, or meditation.

Clause 30. The method of any one of the clauses herein, wherein providing the exercise module comprises providing audio content and/or video content related to the mental aptitude category.

Clause 31. The method of any one of the clauses herein, wherein monitoring the user's performance comprises recording the user's performance via a user device, the method further comprising storing the recorded user's performance in a database.

Clause 32. The method of any one of the clauses herein, wherein monitoring the user's performance comprises recording the user's performance via a user device, the method further comprising providing the recorded user's performance to an assigned coach for review.

Clause 33. The method of any one of the clauses herein, further comprising automatically analyzing the monitored user's performance without human intervention, wherein the provided second test module is based on the analyzed user's performance.

Clause 34. The method of any one of the clauses herein, further comprising analyzing the monitored user's performance using artificial intelligence, wherein the provided second test module is based on the analyzed user's performance.

Clause 35. The method of any one of the clauses herein, wherein the first and/or second test module is based on a review from an assigned coach.

Clause 36. The method of any one of the clauses herein, wherein the exercise module is a first exercise module, the method further comprising:
based on a response to the second test module, determining and providing a second exercise module different than the first exercise module;
monitoring a user's performance of the provided second exercise module; and
based on the monitored performance, providing a third test module different than the first and second test modules and associated with the mental aptitude category.

Clause 37. A non-transitory computer readable medium comprising instructions that, when executed, are configured to perform the method of any one any one of the clauses herein.

### CONCLUSION

Although many of the embodiments are described above with respect to systems, devices, and methods for mental training for sports, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-4F.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for automatically processing a user input for purposes of improving mental skills, the method comprising:
providing a pre-module to a user, the pre-module including a plurality of pre-tagged first objects;
receiving a user input in response to the pre-module provided to the user, the user input including a plurality of second objects;
processing the second objects of the user input by comparing the second objects to the first objects to determine a correlation; and
after processing the second objects, automatically determining a post-module to be provided to the user based on the correlation.

2. The method of claim 1, wherein the correlation corresponds to a percentage of the second objects that match the first objects.

3. The method of claim 2, wherein automatically determining the post-module is based on the match percentage being above a predetermined threshold.

4. The method of claim 1, wherein:
the pre-tagged first objects comprise (i) a subset of primary objects configured to produce a first post-module and (ii) a subset of secondary objects configured to produce a second post-module different than the first post-module,
processing the second objects comprises comparing the second objects to each of the primary objects and the secondary objects, and
automatically determining the post-module comprises automatically determining which of the first post-module or second post-module to provide to the user based on which of the primary objects or the secondary objects has a higher correlation with the second objects.

5. The method of claim 1, wherein automatically determining the post-module is based at least in part on the order of individual second objects relative to one another in the user input.

6. The method of claim 1, wherein the first objects and the second objects comprise keywords, and wherein the second objects are provided in response to a question provided via the pre-module.

7. The method of claim 1, wherein the pre-module is one of a plurality of pre-modules including a first pre-module directed to a first mental aptitude category, and a second pre-module directed to a second mental aptitude category, the second mental aptitude category different than the first mental aptitude category.

8. The method of claim 1, further comprising, after processing the second objects,
if the comparison of the first objects and the second objects is below a predetermined threshold, then sending an indication of such to a system administrator.

9. The method of claim 1, further comprising, after processing the second objects:
if the comparison of the first objects to the second objects is at or above a predetermined threshold, then sending the determined post-module to the user; and
if the comparison of the first objects and the second objects is below a predetermined threshold, then sending an indication of such to a system administrator.

10. The method of claim 1, wherein the pre-module and the post-module are each directed to improving mental skills for a sport.

11. A method for automatically processing user feedback for purposes of developing mental skill, the method comprising:
providing a pre-module to a user;
receiving an indication that the user has completed the pre-module;
after receiving the indication, requesting a user input based at least in part on the provided pre-module;
receiving a user input in response to the pre-module provided to the user, the user input including a plurality of keywords;
processing the keywords of the user input by comparing the keywords to a plurality of pre-tagged keywords to determine a correlation; and
based on the determined correlation, automatically determining a post-module to be provided to the user.

12. The method of claim 11, wherein the post-module is a recommended training instruction comprising at least one of video or audio.

13. The method of claim 11, wherein the steps of providing the pre-module and receiving the user input occur via a mobile application.

14. The method of claim 11, wherein processing the keywords of the user input comprises determining a percentage of the second objects that match the pre-tagged keywords, and wherein automatically determining the post-module is based on the determined percentage being above a predetermined threshold.

15. The method of claim 11, further comprising, after processing the second objects:
if the comparison of the first objects to the second objects is at or above a predetermined threshold, then sending the determined post-module to the user; and
if the comparison of the first objects and the second objects is below a predetermined threshold, then sending an indication of such to a system administrator.

16. A device, comprising:
at least one processor; and
tangible, non-transitory computer-readable media having instructions stored therein, wherein the instructions, when executed by the at least one processor, cause the device to perform operations comprising:
providing a pre-module to a user, the pre-module including a plurality of pre-tagged first objects;
receiving a user input in response to the pre-module provided to the user, the user input including a plurality of second objects;
processing the second objects of the user input by comparing the second objects to the first objects to determine a correlation; and
after processing the second objects, automatically determining a post-module to be provided to the user based on the correlation.

17. The device of claim 16, wherein the correlation corresponds to a percentage of the second objects that match the first objects, and wherein automatically determining the post-module is based on the match percentage being above a predetermined threshold.

18. The device of claim 16, wherein:
the pre-tagged first objects comprise (i) a subset of primary objects configured to produce a first post-module and (ii) a subset of secondary objects configured to produce a second post-module different than the first post-module,
processing the second objects comprises comparing the second objects to each of the primary objects and the secondary objects, and
automatically determining the post-module comprises automatically determining which of the first post-module or second post-module to provide to the user based on which of the primary objects or the secondary objects has a higher correlation with the second objects.

19. The device of claim 16, wherein automatically determining the post-module is based at least in part on the order of individual second objects relative to one another in the user input.

20. The device of claim 16, wherein the post-module is a recommended training instruction comprising at least one of video or audio.
